SEQUENCE LISTING (2 sequences)

SEQ ID NO: 1 - protein, 9 aa, Homo sapiens
RMFPNAPYL

SEQ ID NO: 2 - protein, 8 aa, Homo sapiens
SIINFEKL

(19) United States
(12) Patent Application Publication
(10) Pub. No.: US 2024/0024444 A1
(43) Pub. Date: Jan. 25, 2024

(54) Combined Preparation

(71) Applicant: MEDRx Co., Ltd., Kagawa (JP)

(72) Inventors: Yasushi Miwa, Higashikagawa-shi, Kagawa (JP); Hidetoshi Hamamoto, Higashikagawa-shi, Kagawa (JP); Tatsuhiro Ishida, Tokushima-shi, Tokushima (JP)

(73) Assignee: MEDRx Co., Ltd., Kagawa (JP)

(21) Appl. No.: 18/019,871

(22) PCT Filed: Aug. 6, 2021
(86) PCT No.: PCT/JP2021/029387

§ 371 Date: Feb. 6, 2023

(30) Foreign Application Priority Data
Aug. 7, 2020 (JP) 2020-135294
May 12, 2021 (JP) 2021-081152
Aug. 6, 2021 (WO) PCT/JP2021/029387

(51) Int. Cl. A61K 39/00; A61K 39/39; A61K 47/12; A61K 47/18; A61P 35/00; A61K 9/70

(52) U.S. Cl. CPC A61K 39/001153; A61K 39/39; A61K 47/12; A61K 47/18; A61P 35/00; A61K 9/7023; A61K 2039/55577; A61K 2039/55561; A61K 2039/54

(57) ABSTRACT

The present invention is to provide a combined preparation comprising a first preparation for transdermal administration comprising an antigenic peptide and an aliphatic carboxylic acid-based ionic liquid, and a second preparation for transdermal administration comprising an adjuvant and an aliphatic carboxylic acid-based ionic liquid, enhances the skin permeability of an antigenic peptide and an adjuvant and thus effectively enhances the effect of activating the immune response with the antigenic peptide by the use of the adjuvant.

### TECHNICAL FIELD

The present invention relates to a combined preparation. More specifically, the present invention relates to a combined preparation comprising a first preparation for transdermal administration comprising an antigenic peptide and an aliphatic carboxylic acid-based ionic liquid as well as a second preparation for transdermal administration comprising an adjuvant and an aliphatic carboxylic acid-based ionic liquid.

### BACKGROUND ART

Cancer vaccines are used to induce the cytotoxic T lymphocyte (CTL) for removing cancer cells. The activation of the CTL is achieved by incorporating cancer antigen into the antigen-presenting cell (APC) via major histocompatibility complex (MHC) and presenting the antigen to the CTL. In addition, the APC is required to be activated with an adjuvant to acquire an activated phenotype such as co-stimulatory molecule and cytokine. It has already been reported that there are various types of cancer antigenic peptides and adjuvants that minimize side effects and induce strong and specific immune response to tumors.

Recently, Peptides are focused as antigens for cancer vaccines. Hence, various preparations comprising antigenic peptides are actively studied. Peptides are macromolecules with high water solubility, and thus the administration route thereof has been limited to oral administration or administration by injection such as intramuscular administration and subcutaneous administration. On the other hand, the administration methods have any disadvantages. For example, oral administration has any significant impact on the drug efficacy due to the gastrointestinal absorption and the first-pass effects, and administration by injection causes patients pain and produces serious side effects. As for adjuvants used with antigens, the administration route of the adjuvants when used with peptides has also been limited to oral administration or administration by injection.

Hence, various administration routes other than oral administration and administration by injection have been studied. Skin has been considered as an attractive administration site for vaccines because of its relative ease of use and the high presence rate of APCs. The epidermis and dermis, which are located below the stratum corneum in skin tissues, are rich in Langerhans cells (LC) and dermal dendritic cells (dDC) which are APCs. These cells migrate to the lymph nodes and capture an antigen to present the antigen. Among them, Langerhans cells have a high effect for priming CD8⁺ T cells. Hence, it has been desired to develop a preparation for transdermal administration suitable for the delivery to Langerhans cells. On the other hand, compounds with a molecular weight of 500 Da or more such as cancer antigenic peptide consisting of 9 amino acid residues which basically corresponds to a compound with a molecular weight of about 1000 Da are blocked in their delivery by the stratum corneum having a strong skin barrier function, and thus there was the problem that cancer antigenic peptides and adjuvants were not sufficiently delivered to the APCs for the conventional cancer vaccines. Hence, it was required to use transdermal absorption enhancers for enhancing the permeability of cancer antigenic peptide to the stratum corneum.

Patent Document 1 discloses a cancer vaccine tape preparation for transdermal administration comprising a WT1 peptide, which is cancer antigenic peptide, and a cellular immunity induction promoter such as a TLR ligand and a cyclic dinucleotide. Also, it is disclosed that a skin permeability enhancer such as higher alcohols, polyhydric alcohols and higher fatty acids is used to improve the skin permeability of the transdermally-administered WT1 peptide. Patent Document 2 discloses a cancer vaccine composition for transdermal administration comprising a WT1 peptide and an organic acid such as lactic acid, salicylic acid, citric acid and methanesulfonic acid as a cellular immunity induction promoter. Also, it is disclosed that the organic acid is used to improve the induction of cellular immunity to an antigen. In the compositions for transdermal administration, all ingredients such as cancer antigenic peptides are contained in the same composition.

It has not been reported that a cancer antigenic peptide and an adjuvant are separately prepared in each preparation and also that they are separately dissolved in an aliphatic carboxylic acid-based ionic liquid.

Peptides are also recently focused as vaccines for diseases other than cancer, and the techniques for enhancing the permeability of antigenic peptides into the stratum corneum when the peptides are used as preparations for transdermal administration are needed. However, it has not been reported that the peptides other than cancer antigenic peptides are separately prepared in a different preparation from adjuvants and also that such peptides and adjuvants are separately dissolved in an aliphatic carboxylic acid-based ionic liquid.

### PRIOR ART DOCUMENTS

### Patent Documents

Patent Document 1: JP 2014-169279
Patent Document 2: JP 2014-169276

### SUMMARY OF INVENTION

### Problem to be Solved by the Invention

An object of the present invention is to provide a preparation for transdermal administration which enhances the skin permeability of an antigenic peptide and an adjuvant and thus effectively enhances the effect of activating the immune response with the antigenic peptide by the use of the adjuvant.

### Means for Solving the Problems

The present inventors have extensively studied to reach the object, and have found that the skin permeability of both an antigenic peptide and an adjuvant is improved by not administering a preparation comprising both an antigenic peptide and an adjuvant but preparing each preparation for transdermal administration in which an antigenic peptide and an adjuvant are separately dissolved in an aliphatic carboxylic acid-based ionic liquid, which acts as a transdermal absorption enhancer and administering the preparations in combination. In addition, the present inventors have found that when cancer antigenic peptide is used as an antigenic peptide, the cancer antigenic peptide remarkably produces the effect of inducing cytotoxic T lymphocytes (CTLs) and thus produces an effect of inhibiting the growth of tumor when the combined preparation is administered to mice transplanted with cancer cells. Based upon the new findings, the present invention has been completed.

That is, the present invention provides the following embodiments.
[Item 1] A combined preparation comprising a first preparation for transdermal administration comprising an antigenic peptide and an aliphatic carboxylic acid-based ionic liquid, and a second preparation for transdermal administration comprising an adjuvant and an aliphatic carboxylic acid-based ionic liquid.
[Item 2] The combined preparation according to the item 1, wherein the second preparation for transdermal administration is applied to the skin and then the first preparation for transdermal administration is applied to the same site.
[Item 3] The combined preparation according to the item 1 or 2, wherein the antigenic peptide is cancer antigenic peptide.
[Item 4] The combined preparation according to the item 3, wherein the cancer antigenic peptide is WT1 peptide.
[Item 5] The combined preparation according to any one of the items 1 to 4, wherein the adjuvant is Resiquimod, Poly-IC or Quil-A.
[Item 6] The combined preparation according to any one of the items 1 to 5, wherein the aliphatic carboxylic acid-based ionic liquid is an aliphatic carboxylic acid-based mixed ionic liquid.
[Item 7] The combined preparation according to the item 6, wherein the aliphatic carboxylic acid-based mixed ionic liquid is prepared from a) one or more lower aliphatic carboxylic acid-based ionic liquids selected from salts of ethanolamine, diethanolamine or triethanolamine with a short-chain fatty acid and b) an aliphatic carboxylic acid-based ionic liquid having 2 to 20 carbon atoms.
[Item 8] The combined preparation according to any one of the items 1 to 7, wherein each of the first and second preparations for transdermal administration further comprises an excipient.
[Item 9] The combined preparation according to any one of the items 1 to 8, which is a preparation for vaccine.
[Item 10] The combined preparation according to any one of the items 3 to 9, which is an agent for inducing cytotoxic T lymphocytes.
[Item 11] Use of a first preparation for transdermal administration comprising an antigenic peptide and an aliphatic carboxylic acid-based ionic liquid as well as a second preparation for transdermal administration comprising an adjuvant and an aliphatic carboxylic acid-based ionic liquid in the manufacture of an agent for vaccine.
[Item 12] The use according to the item 11, wherein the agent for vaccine is an agent for cancer vaccine and the antigenic peptide is cancer antigen peptide.
[Item 13] A kit of preparation for transdermal administration comprising:
   1) a first preparation for transdermal administration comprising an antigenic peptide and an aliphatic carboxylic acid-based ionic liquid;
   2) a second preparation for transdermal administration comprising an adjuvant and an aliphatic carboxylic acid-based ionic liquid; and
   3) instructions for use for administering the above 1) and 2) in combination.
[Item 14] The kit according to the item 13 for use as cancer vaccine, wherein the antigenic peptide is cancer antigen peptide.

In addition, the present invention provides the following embodiments.
[Item 15] A method of administering a peptide vaccine to a patient in need thereof, which comprises administering a first preparation for transdermal administration comprising a therapeutically effective amount of an antigenic peptide and an aliphatic carboxylic acid-based ionic liquid in combination with a second preparation for transdermal administration comprising an adjuvant and an aliphatic carboxylic acid-based ionic liquid to the patient.
[Item 16] The method according to the item 15, wherein the second preparation for transdermal administration is administered and then the first preparation for transdermal administration is administered.
[Item 17] The combined preparation according to any one of the items 3 to 10 for use in the treatment of cancer.
[Item 18] A preparation for transdermal administration comprising an adjuvant and an aliphatic carboxylic acid-based ionic liquid, wherein the preparation is used in combination with a preparation for transdermal administration comprising an antigenic peptide and an aliphatic carboxylic acid-based ionic liquid.
[Item 19] The preparation for transdermal administration according to the item 18, wherein the antigenic peptide is cancer antigenic peptide.
[Item 20] A preparation for transdermal administration comprising an antigenic peptide and an aliphatic carboxylic acid-based ionic liquid, wherein the preparation is used in combination with a preparation for transdermal administration comprising an adjuvant and an aliphatic carboxylic acid-based ionic liquid.
[Item 21] The preparation for transdermal administration according to the item 20, wherein the antigenic peptide is cancer antigenic peptide.
[Item 22] A method of activating the immune response with an antigenic peptide, which comprises administering a first preparation for transdermal administration comprising a therapeutically effective amount of an antigenic peptide and an aliphatic carboxylic acid-based ionic liquid in combination with a second preparation for transdermal administration comprising an adjuvant and an aliphatic carboxylic acid-based ionic liquid to a patient in need thereof.
[Item 23] A method of treating cancer, which comprises administering a first preparation for transdermal administration comprising a therapeutically effective amount of an antigenic peptide and an aliphatic carboxylic acid-based ionic liquid in combination with a second preparation for transdermal administration comprising an adjuvant and an aliphatic carboxylic acid-based ionic liquid to a patient in need thereof.

### Effects of the Invention

According to the present invention, an antigenic peptide and an adjuvant effectively penetrate the skin and thus the antigenic peptide can be presented to antigen presenting cells (APCs) to significantly activate the immune response. For example, when cancer antigenic peptide is used as an antigenic peptide, the remarkable induction of cytotoxic T lymphocytes can be achieved systemically and thus the peptide can be used in the treatment of cancer such as skin cancer and lung cancer. In addition, the effect of enhancing the immune response with an adjuvant can be improved by administering a preparation for transdermal administration comprising an antigenic peptide and a preparation for transdermal administration comprising an adjuvant separately, and thus the effect of activating the immune response with the antigenic peptide can be further enhanced. The combined preparation of the present invention is expected as a preparation for transdermal administration for use in vaccines.

### BRIEF DESCRIPTION OF THE DRAWINGS

FIG. 1 shows the changes in the accumulative skin permeation amounts of WT1 peptide and R848 in Preparation Examples 1 to 3. In FIG. 1, (a) shows the change in the accumulative skin permeation amount of R848 in Preparation Example 1, (b) shows the change in the accumulative skin permeation amount of WT1 peptide in Preparation Example 1, (c) shows the change in the accumulative skin permeation amount of R848 in Preparation Example 3, and (d) shows the change in the accumulative skin permeation amount of WT1 peptide in Preparation Example 2.

FIG. 2 shows ratios of cytotoxic T lymphocytes (CTLs) in spleen cells after the treatment with the control, the WT1 peptide/R848 preparations applied to abdomen (TS (−) and TS (+)) and back (TS (−) and TS (+)) and WT1/CFA preparation which is the positive control (%). TS (−) shows that tape stripping treatment is not included, and TS (+) shows that tape stripping treatment is included.

FIG. 3 shows the ratios of white blood cells and dendritic cells after 0 day, 1 day and 4 days of the application of R848 preparation (%). In FIG. 3, (a) shows the ratio of white blood cells (%), and (b) shows the ratio of dendritic cells (%).

FIG. 4 shows the ratios of cytotoxic T lymphocytes (CTLs) in spleen cells after the administration of the ionic liquid-containing preparation comprising WT1 peptide/R848 of Example 3 as well as the combined preparation of the ionic liquid-containing preparation comprising WT1 peptide and the ionic liquid-containing preparation comprising R848 of the present invention (%).

FIG. 5 shows the results of the measured tumor volume of mice in the control group, the I-OHP-containing liposome treatment group, the WT1 peptide/R848 preparation treatment group and the combined preparation of WT1 peptide preparation and R848 preparation treatment group (mm³). In FIG. 5, (a) shows the results of the control group, the I-OHP-containing liposome treatment group and the WT1 peptide/R848 preparation treatment group, and (b) shows the results of the control group and the combined preparation of WT1 peptide preparation and R848 preparation treatment group.

FIG. 6 shows the results of the measured tumor volume of mice in the control group, the I-OHP-containing liposome treatment group, the OVA peptide/R848 preparation treatment group and the combination of I-OHP-containing liposome and OVA peptide/R848 preparation treatment group (mm³).

FIG. 7 shows the results of the measured tumor volume of mice in the control group, the OVA peptide/R848 preparation treatment group, the combined preparation of OVA peptide preparation and R848 preparation treatment group and the combined preparation of R848 preparation and OVA peptide preparation treatment group (mm³).

FIG. 8 shows the results of the ratio of immune cells in the skin tissue (%) and the antigen presentation level of OVA peptide (Mean Fluorescence Intensity; MFI) in the treatment with the combined preparation of R848 preparation and OVA peptide preparation. In FIG. 8, (a) shows the ratio of white blood cells (%), (b) shows the ratio of dendritic cells (%), (c) shows the antigen presentation level of OVA peptide in white blood cells (MFI), and (d) shows the antigen presentation level of OVA peptide in dendritic cells (MFI).

FIG. 9 shows the results of the ratios of immune cells in lymph nodes (%), the antigen presentation levels of OVA peptide in immune cells (MFI) and the expression levels of CD86 in immune cells (MFI) in the control (untreatment) and the treatment with the combined preparation of R848 preparation and OVA peptide preparation. In FIG. 9, (a) shows the ratio of macrophage cells (%), (b) shows the ratio of dendritic cells (%), (c) shows the antigen presentation level of OVA peptide in macrophage cells (MFI), (d) shows the antigen presentation level of OVA peptide in dendritic cells (MFI), (e) shows the expression level of CD86 in macrophage cells (MFI), and (f) shows the expression level of CD86 in dendritic cells (MFI).

FIG. 10 shows the ratios of each type of immune cells (macrophage cells, dendritic cells and granulocytes) in CD45 positive cells in the skin tissue in the control (untreatment) and the treatment with each preparation comprising an adjuvant (R848, Poly-IC or Quil-A) (%).

### DESCRIPTION OF EMBODIMENTS

The present invention provides a combined preparation comprising a first preparation for transdermal administration comprising an antigenic peptide and an aliphatic carboxylic acid-based ionic liquid as well as a second preparation for transdermal administration comprising an adjuvant and an aliphatic carboxylic acid-based ionic liquid.

As used herein, the “antigenic peptide” is not particularly limited as long as it induces the immune response in the body of a subject to be administered. Also, the sequence of the antigenic peptide and the length thereof are not particularly limited. For example, a peptide having 2 to 50 amino acids, a peptide having 2 to 30 amino acids, a peptide with a molecular weight of 5000 or less, or a peptide with a molecular weight of 3000 or less is preferable.

Examples of the antigenic peptide of the present invention include a peptide derived from pathogens such as bacteria, fungi and viruses; cancer antigenic peptide; a peptide derived from peptide hormones, cytokines, growth factors and receptor proteins thereof, but are not limited thereto.

Examples of the antigenic peptide of the present invention also include a fragment of OVA peptide, dengue virus DEN3-ED3, gag and poly of human hepatitis virus or influenza virus, BAGE, CASP8, CEA, Her2/neu, MAGE-1, MAGE-3, MAGE-A4, MART1, MUC1, NY-ESO-1, p53, PSA, PRAME, TRP1, TRP2, ras, SART-1, IFN-α, IL-6, IL-12, IL-17 and IL-23.

The OVA peptide is an immunogenic peptide derived from an albumin from egg white which is an egg allergen. For example, the OVA peptide is a MHC-restricted peptide having the amino acid sequence of SEQ ID NO: 2. Examples of the OVA peptide include OVA Peptide (257-264) and OVA Peptide (323-339).

As used herein, the term “cancer antigenic peptide” refers to an peptide that can induce and/or activate the CTLs, recognized by cancer-specific cytotoxic T cells (CTLs) from tissue or body fluids or cells of a mammalian organism or from antigen-presenting cells derived from a mammalian organism. The cancer antigenic peptide may be bound to transmembrane peptide receptors comprising MHC class I and MHC class II molecules presenting the antigenic peptide at the cell surface to T cells of the immune system. The caner antigenic peptide may also be bound to intra- or extracellular MHC molecules. The caner antigenic peptide may also be bound to intracellular peptide receptors relating to the heat shock protein (Hsp) family. The cancer antigenic peptide may be a peptide having the sequence in which one or two amino acids are added, substituted or deleted in the amino acid sequence.

Examples of the cancer antigenic peptide include a peptide derived from proteins such as WT1, PR1, GPC3, HER-2, MAGE-A1, MAGE-A2, MAGE-A3, gp100, CEA, hTRT, mTERT, PRAME, PSMA, PSA-1 and MUC-1, but are not limited thereto.

The cancer antigenic peptide of the present invention is preferably a WT1 peptide and an OVA peptide. The WT1 peptide is a peptide which is a fragmented peptide of WT1 protein, which is a product of oncogene WT1 (Wilm's tumor), and has the amino acid sequence of SEQ ID NO: 1 consisting of human WT1 protein-derived contiguous amino acids. For example, the cancer antigenic peptide retains the ability to bind MHC class I or MHC class II molecule and has the ability to induce WT1-specific killer or helper T cells. The amino acid sequence and length of the WT1 peptide of the present invention are not particularly limited as long as the peptide has said feature. The length of the WT1 peptide of the present invention is preferably 10 to 25 amino acids, more preferably 15 to 21 amino acids and furthermore preferably 16 to 20 amino acids, and may be, for example, 17 amino acids, 18 amino acids or 19 amino acids. The antigenic peptide of the present invention also includes peptides such as db126 peptide, db221 peptide and db235 antigenic peptide.

The antigenic peptide of the present invention may be in the free form or in any pharmacologically acceptable salt form such as an acid salt (e.g., acetic acid salt, TFA salt, hydrochloric acid salt, sulfuric acid salt, phosphoric acid salt, lactic acid salt, tartaric acid salt, maleic acid salt, fumaric acid salt, oxalic acid salt, hydrobromic acid salt, succinic acid salt, nitric acid salt, malic acid salt, citric acid salt, oleic acid salt, palm itic acid salt, propionic acid salt, formic acid salt, benzoic acid salt, picric acid salt, benzenesulfonic acid salt, dodecyl sulfuric acid salt, methanesulfonic acid salt, p-toluenesulfonic acid salt, glutaric acid salt and salts of each amino acid), a metal salt (e.g., alkali metal salt (e.g., sodium salt, potassium salt), alkali earth metal salt (e.g., calcium salt, magnesium salt) and aluminum salt), an amine salt (e.g., triethylamine salt, benzylamine salt, diethanolamine salt, t-butylamine salt, dicyclohexylamine salt, arginine salt, dimethylammonium salt and ammonium salt).

The antigenic peptide of the present invention may be synthesized or generated, isolated, and purified according to any method well known in the art.

As used herein, the term “adjuvant” refers to a substance which enhances the immune response to an antigen. Examples of the adjuvant include Freund's incomplete adjuvant, BCG, trehalose dimycolate, lipopolysaccharide, alum adjuvant and silica adjuvant.

As the adjuvant of the present invention, an adjuvant to a WT1 peptide may be used. Examples thereof include mineral gel; lysolecithin, Pluronic polyol; polyanion; peptide; and oil emulsion, and GM-CSF, BCG-CWS and Montanide. Also, various vaccine adjuvants are further preferable. Examples thereof include imidazoquinolines such as Imiquimod, R848 (Resiquimod), 1H-imidazo[4,5-c]quinolin-4-amine, cyclic dinucleotides such as cyclic diguanosine monophosphate, nucleic acid adjuvants (nucleic acid-based immunoadjuvants) such as ligands recognized by various TLRs (such as TLR 2, 3, 7, 8 and 9), for example, CPG DNA (ligand recognized by TLR9), POLY-IC (ligand recognized by TLR3) and saponin adjuvants such as Quil-A, Matrix-M®, Abisco®, AS01B and QS21. Among them, Resiquimod, POLY-IC and Quil-A are particularly preferable.

As used herein, the term “aliphatic carboxylic acid-based ionic liquid” refers to a Brønsted salt prepared from an aliphatic carboxylic acid and an organic cation, which is in a viscous liquid form at ambient temperature and is a melting point of 100° C. or less. The aliphatic carboxylic acid-based ionic liquid may be prepared by mixing an aliphatic carboxylic acid and an organic cation in equimolar amounts or excess amounts at room temperature or with heating. The excess amounts of the aliphatic carboxylic acid and/or organic cation are preferably within 50 time molar amounts. Also, the aliphatic carboxylic acid-based ionic liquid of the present invention may be prepared from a drug having an aliphatic carboxylic acid and an amine structure.

As used herein, the term “aliphatic carboxylic acid” refers to a carboxylic acid having one or more carboxyl groups. The carbon chain in the aliphatic carboxylic acid may be straight or branched and saturated or unsaturated. In addition, the aliphatic carboxylic acid may have a group(s) other than carboxyl group, and the number and type of the group(s) are not particularly limited. Examples of the group(s) include amino group, hydroxyl group.

Examples of the aliphatic carboxylic acid include an aliphatic carboxylic acid having 2 to 20 carbon atoms. Examples of the aliphatic carboxylic acid having 2 to 20 carbon atoms include an aromatic carboxylic acid having 7 to 9 carbon atoms, a short-chain fatty acid and a keto acid having 2 to 7 carbon atoms, a medium-chain fatty acid having 8 to 12 carbon atoms and a higher fatty acid having 13 to 20 carbon atoms.

Examples of the aliphatic carboxylic acid of the present invention include salicylic acid, lactic acid, glycolic acid, methoxyacetic acid, levulinic acid, hexanoic acid, 2-ethylhexanoic acid, octanoic acid, decanoic acid, lauric acid, myristic acid, palmitic acid, stearic acid, isostearic acid and oleic acid. Among them, salicylic acid, lactic acid and isostearic acid are preferable.

As used herein, the term “organic cation” refers to a cationic organic compound, and examples thereof include an organic amine, an organic quaternary ammonium cation, an organic quaternary phosphonium cation. Examples of the organic amine include an organic amine having 4 to 12 carbon atoms. Among them, ethanolamine, diethanolamine, triethanolamine, isopropanolamine, diisopropanolamine and triisopropanolamine are preferable.

The aliphatic carboxylic acid-based ionic liquid as used herein may be an aliphatic carboxylic acid-based mixed ionic liquid. As used herein, the term “aliphatic carboxylic acid-based mixed ionic liquid” refers to an ionic liquid prepared by mixing two or more of said aliphatic carboxylic acid-based ionic liquids (e.g., aliphatic carboxylic acid-based ionic liquids having 2 to 20 carbon atoms), and encompasses three types of mixed ionic liquids which are mixed ionic liquids prepared by using a common aliphatic carboxylic acid and a different organic cation (organic amine), mixed ionic liquids prepared by using a common organic cation (organic amine) and a different aliphatic carboxylic acid, and mixed ionic liquids prepared by using a different aliphatic carboxylic acid and a different organic cation (organic amine). Examples thereof include an aliphatic carboxylic acid-based mixed ionic liquid prepared from a) one or more aliphatic carboxylic acid-based ionic liquids having 2 to 7 carbon atoms prepared from a lower aliphatic carboxylic acid having 2 to 7 carbon atoms and any of ethanolamine, diethanolamine or triethanolamine and b) an aliphatic carboxylic acid-based ionic liquid having 2 to 20 carbon atoms.

In the present invention, an aliphatic carboxylic acid-based ionic liquid in which the solubilities of an antigenic peptide and an adjuvant are 1 w/w % or more may be used. Preferred examples of the aliphatic carboxylic acid-based ionic liquid include a salt of diisopropanolamine or with the aliphatic carboxylic acid-based ionic liquid having 2 to 7 carbon atoms and a salt of triisopropanolamine with the aliphatic carboxylic acid-based ionic liquid having 2 to 7 carbon atoms.

The combined preparation of the present invention may be used in the prophylaxis or treatment of various diseases depending on the type of antigenic peptide therein. Examples of such disease include various types of infectious diseases.

As used herein, the term “infectious disease” refers to a disease caused by the infection of the host with pathogens such as bacteria, fungi, viruses and parasites.

The combined preparation of the present invention may be used in the cancer vaccine therapy as a preparation for vaccine and an agent for inducing cytotoxic T lymphocytes as long as cancer antigenic peptide is used as an antigenic peptide. The cancer vaccine therapy may be used in the prophylaxis or treatment of cancer.

As used herein, the term “agent for inducing cytotoxic T lymphocytes” refers to an agent for producing the immunostimulatory effect against cancer cells or pathogens in vivo or in vitro by differentiating and/or activating cytotoxic T-lymphocytes that specifically recognize the cancer antigenic peptide of the present invention.

As used herein, the “cancer” includes solid cancer, blood cancer, metastatic cancer, but the type of cancer is not particularly limited. The metastasis of cancer includes hematogenous metastasis, lymphatic metastasis and disseminated metastasis. Examples of the solid cancer include brain tumor, lung cancer, gastric cancer, colorectal cancer, liver cancer, pancreatic cancer, renal cancer, adrenal cancer, gallbladder/biliary tract cancer, esophageal cancer, pharyngeal cancer, laryngeal cancer, oral cancer, bladder cancer, cancer of the renal pelvis and ureter, tongue cancer, thyroid cancer, skin cancer, breast cancer, prostate cancer, testicular cancer, uterine cancer, cervical cancer, ovarian cancer, bone musculoskeletal tumor, osteosarcoma, chondrosarcoma, rhabdomyosarcoma and leiomyosarcoma. Examples of the blood cancer include multiple myeloma, malignant lymphoma (e.g., Non-Hodgkin's lymphoma, Hodgkin's lymphoma) and leukemia (e.g., acute myeloid leukemia, chronic myeloid leukemia). Examples of the metastatic cancer include metastatic brain tumor, metastatic lung cancer, metastatic gastric cancer, metastatic colorectal cancer, metastatic liver cancer, metastatic pancreatic cancer, metastatic renal cancer, metastatic adrenal cancer, metastatic esophageal cancer, metastatic bladder cancer, metastatic thyroid cancer, metastatic breast cancer, metastatic prostate cancer, metastatic uterine cancer and metastatic osteosarcoma. Preferably, cancer is cancer associated with WT1 gene.

The “prophylaxis” as used herein is intended to prevent the development of a disease, delay the development of a disease and reduce the risk of developing a disease. As used herein, the term “treatment” refers to all types of treatments of a disease. For example, the treatment when the disease is cancer is intended to completely remove cancer tissues, kill cancer cells, inhibit the growth of cancer cells, reduce the symptoms associated with cancer, improve the quality of life for cancer patients, and prolong the survival of cancer patients. Also, the treatment encompasses the prevention of the recurrence of cancer.

As used herein, the term “patient” refers to human and an animal such as dog, cat and horse. Among them, human is preferable.

As used herein, the term “therapeutically effective amount” refers to an amount that can produce the effects of treating a disease or an amount that can delay the progression of the disease, compared with untreated patients. The term also encompasses, within its scope, an amount effective to promote normal physiological functions. The effective amount includes the amount of the antigenic peptide of the present invention useful for the treatment of a disease or the amount of the antigenic peptide of the present invention in combination with other active ingredient(s) useful fora disease.

The first preparation for transdermal administration in the combined preparation of the present invention may comprise a pharmaceutically acceptable active ingredient other than an antigenic peptide. For example, when an antigenic peptide is cancer antigenic peptide, the first preparation may comprise an anti-tumor agent.

Each preparation in the combined preparation of the present invention may comprise commonly-used known additive(s), for example, an antioxidant, a preservative, a thickener. The additive may be used alone, or two or more of the additives may be combined in appropriate amounts.

Examples of the antioxidant include a water-soluble antioxidant and a hydrophobic antioxidant. Examples thereof include ascorbic acid, sodium hydrogen sulfite, sodium sulfite, erythorbic acid, tocopherol acetate, dibutylhydroxytoluene, tocopherol, sodium pyrosulfite, butylhydroxyanisole and propyl gallate. The amount of the antioxidant in each preparation in the combined preparation of the present invention may be appropriately adjusted depending on, for example, the type of the antioxidant. The amount is, for example, 0.01 to 1% by weight relative to the total amount of the drug layer. Also, the antioxidant may be used alone, or two or more of the antioxidants may be used in combination.

Examples of the preservative include benzoic acid, sodium benzoate, sorbic acid, sodium sorbate, sodium dehydroacetate, parahydroxybenzoic acid, sodium parahydroxybenzoate, ethyl parahydroxybenzoate, propyl parahydroxybenzoate (propylparaben), butyl parahydroxybenzoate, isopropyl parahydroxybenzoate, isobutyl parahydroxybenzoate, propionic acid, sodium propionate, benzalkonium chloride and salicylic acid. Among them, methylparaben, propylparaben, benzalkonium chloride, salicylic acid or a mixture thereof is preferable. The amount of the preservative in each preparation in the combined preparation of the present invention may be appropriately adjusted depending on, for example, the type of the preservative. The amount is, for example, 0.01 to 1% by weight relative to the total amount of the drug layer. Also, the preservative may be used alone, or two or more of the preservatives may be used in combination.

The thickener encompasses an inorganic material and an organic material. Examples of the inorganic material include amorphous silicon dioxide, kaolin (gypsum), diatomite, talc, hydrated silicon dioxide, light anhydrous silicic acid, magnesium silicate, calcium silicate, calcium phosphate and barium sulfate, and examples of the organic material include crystalline cellulose. The amount of the thickener in each preparation in the combined preparation of the present invention may be appropriately adjusted depending on, for example, the type of the thickener. The amount is, for example, 0.01 to 1% by weight relative to the total amount of the drug layer. Also, the thickener may be used alone, or two or more of the thickeners may be used in combination.

The combined preparation of the present invention is suitable for transdermal administration. The dosage form of the combined preparation of the present invention may be an external preparation such as ointment, cream, gel and patch and prepared according to any conventional method. In addition, the combined preparation of the present invention may be transdermally administrated using microneedles.

Each preparation in the combined preparation of the present invention may be prepared by dissolving an antigenic peptide or an adjuvant in an aliphatic carboxylic acid-based mixed ionic liquid, dissolving the ionic liquid in a base of the preparation or mixing and dispersing the ionic liquid and the base, and suspending the solution. Examples of the base include bases used in various preparations such as ointment, solution and patch.

Examples of the base for ointment include white petrolatum, liquid paraffin and gelled hydrocarbon. The gelled hydrocarbon means substances in which hydrocarbons are gelled such as liquid paraffin, paraffin, isoparaffin, squalane, squalene and polybuten. The gelled hydrocarbon in which liquid paraffin is gelled with polyethylene resin and the gelled hydrocarbon in which an oil is gelled with rubber elastomer are particularly preferable. Examples thereof include Plastibase (trade name) in which liquid paraffin (the Japanese Pharmacopoeia) is gelled with 5 to 10% by weight of polyethylene resin, Poloid (trade name), hydrophilic gelled hydrocarbon in which glycerin fatty acid ester is added to gelled hydrocarbon to give hydrophilicity (Plastibase Hydrophilic (trade name)).

Examples of the base for solution include a mixture solution of an alcohol such as isopropanol, ethanol, propylene glycol, glycerin and an oil such as olive oil and soybean oil.

Examples of the base for patch include an adhesive. The adhesive as the base for patch mainly comprises an elastomer and a tackifier, a softening agent, a filler, an antioxidant and the like. The adhesive may not comprise a softening agent, a filler and an antioxidant.

The combined preparation of the present invention may be provided as a kit of preparation for transdermal administration. For example, the kit comprises a preparation comprising an antigenic peptide and a preparation comprising an adjuvant. The kit of the present invention may be provided together with a package insert, a packaging container and instructions for use indicating matters such as the usage and dose of an antigenic peptide and an adjuvant in their combined use. In a certain embodiment, the kit of the present invention may be provided as a medical product for the treatment of cancer.

The amounts to be used of each preparation in the combined preparation of the present invention vary with various factors such as the symptom and the age of patients. In general, the preparations are administered to adults once to several times daily.

The combined preparation of the present invention may be prepared in separate dosage forms and administered separately. Also, in the combined preparation of the present invention, the first preparation for transdermal administration may be administered before, simultaneously with, or after the administration of the second preparation for transdermal administration. For example, the combined preparation of the present invention may be administered by applying the second preparation for transdermal administration to the skin and then administering the first preparation for transdermal administration intradermally or subcutaneously or by applying the first preparation for transdermal administration to the skin and then administering the second preparation for transdermal administration intradermally or subcutaneously.

The combined preparation of the present invention may be administered at the same or different sites.

### EXAMPLES

Hereinafter, the present invention is described more specifically with reference to Examples. However, the present invention is not intended to be limited to them by any means.

### Example 1: Preparation of Test Preparation

### (1) Preparation of Ionic Liquid-Containing Preparation

Each ingredient was weighed in the amounts shown in Table 1 below, and the preparations of Preparation Examples 1 to 5 were prepared. The preparation of Preparation Example 1 was prepared by mixing lactic acid, isostearic acid and triethanolamine to prepare an ionic liquid, dissolving the WT1 peptide (MHC-restricted peptide: RMFPNAPYL (SEQ ID NO: 1), purchased from SCRUM Inc.) and Resiquimod (R848: purchased from R&D Systems (Minneapolis, MN)) into the prepared ionic liquid, and then adding tartaric acid, glycerin, dimethyl isosorbide, isopropyl myristate, 2-propanol and water thereto.

The preparation of Preparation Example 2 was prepared by mixing lactic acid, isostearic acid and triethanolamine to prepare an ionic liquid, dissolving the WT1 peptide into the prepared ionic liquid and then adding an ionic liquid consisting of diisopropanolamine and salicylic acid prepared by pre-mixing them thereto and mixing them, and then adding dimethyl isosorbide, isopropyl myristate, glycerin, propylene glycol, propylene carbonate, Macrogol 400 and 2-propanol thereto.

The preparation of Preparation Example 3 was prepared by mixing lactic acid, isostearic acid and triethanolamine to prepare an ionic liquid, dissolving Resiquimod into the prepared ionic liquid, and then adding tartaric acid, dimethyl isosorbide, glycerin, propylene glycol, isopropyl myristate, 2-propanol and water thereto.

The preparation of Preparation Example 4 was prepared by mixing lactic acid, isostearic acid and triethanolamine to prepare an ionic liquid, dissolving the OVA peptide (MHC-restricted peptide: SIINFEKL (SEQ ID NO: 2), purchased from MEDICAL & BIOLOGICAL LABORATORIES CO., LTD. (MBL)) and Resiquimod into the prepared ionic liquid, and then adding tartaric acid, glycerin, dimethyl isosorbide, isopropyl myristate, 2-propanol and water thereto.

The preparation of Preparation Example 5 was prepared by lactic acid, isostearic acid and triethanolamine to prepare an ionic liquid, dissolving the OVA peptide into the prepared ionic liquid and then adding an ionic liquid consisting of diisopropanolamine and salicylic acid prepared by pre-mixing them thereto and mixing them, and then adding dimethyl isosorbide, isopropyl myristate, glycerin, propylene glycol, propylene carbonate, Macrogol 400 and 2-propanol thereto.

TABLE 1

| | Preparation | Preparation | Preparation | Preparation | Preparation |
|---|---|---|---|---|---|
| | Ex.1 | Ex.2 | Ex.3 | Ex.4 | Ex.5 |
| WT1 peptide | 5 | 5 | | | |
| OVA peptide | | | | 5 | 5 |
| Resiquimod (R848) | 5 | | 5 | 5 | |
| triethanolamine | 10 | 35 | 2 | 10 | 35 |
| lactic acid | 5 | 85 | 10 | 5 | 85 |
| isostearic acid | 20 | 55 | 18 | 20 | 55 |
| tartaric acid | 5 | | 5 | 5 | |
| dimethyl isosorbide | 90 | 90 | 90 | 90 | 90 |
| isopropyl myristate | 295 | 200 | 260 | 295 | 200 |
| glycerin | 5 | 30 | 5 | 5 | 30 |
| propylene glycol | | 290 | 100 | | 290 |
| propylene | | 50 | | | 50 |
| carbonate | | | | | |
| diisopropanolamine | | 30 | | | 30 |
| salicylic acid | | 5 | | | 5 |
| macrogol400 | | 20 | | | 20 |
| 2-propanol | 270 | 105 | 400 | 270 | 105 |
| water | 290 | | 100 | 290 | |
| Total | 1000 | 1000 | 995 | 1000 | 1000 |

### (2) Preparation of I-OHP Containing Liposome

In the preparation of an I-OHP containing liposome, oxaliplatin (I-OHP) containing liposome consisting of HSPC/Chol/mPEG₂₀₀₀-DSPE (molar ratio: 2/1/0.2) was prepared by the reverse-phase evaporation method. Non-encapsulated I-OHP was dialyzed and removed using dialysis cassettes (Slide A-Lyzer, 10000MWCO; Thermo Fisher Scientific, MA, USA) for 5% dextrose solution. The liposome was destroyed in 1% Triton-X solution, and then the concentration of I-OHP in the liposome was quantified by atomic absorption photometer (Z-5700 series, Hitachi High-Tech Science Corporation). The concentration of phospholipids in the liposome was quantified by phosphorus assay. The average diameter of the liposome is about 100 nm, which was measured by Zetasizer Nano (Malvern Instruments, UK). HSPC (hydrogenated soybean phosphatidylcholine) and mPEG₂₀₀₀-DSPE (2-distearoyl-sn-glycero-3-phosphoethanolamine-n-[methoxy(polyethyleneglycol)-2000]) were purchased from NOF (Tokyo), and Chol (cholesterol) was purchased from FUJIFILM Wako Pure Chemical Corporation. All lipids was used without any specific purification.

### Example 2: In Vitro Skin Permeability Test

In this test, a Franz diffusion cell (effective diffusion area: 1.0 cm², PermeGear, Hellertown, US) was used, and the abdominal skin (shaved before the skin is removed) of 5-week-old male rats (Slc: Wistar) was used. The preparation of Preparation Examples 1 to 3 were used as test preparations.

According to the following procedure, the skin permeation amounts of each ingredient in the preparations of Preparation Examples 1 to 3 were measured.

The abdominal skin of rats was cut to about 8.5 cm² (23 mm round) and set in a receptor cell filled with 8.0 mL of the receptor solution (saline of 0.1% bovine serum albumin (BSA) (purchased from FUJIFILM Wako Pure Chemical Corporation)). The receptor solution was kept at 32° C. in a circulator water bath at a constant temperature. After 2, 4, 6, 8 and 24 hours, the receptor solution (0.2 mL) was collected and each of the collected solution was used as samples. After collecting the samples, the same amount of the receptor solution was added into the receptor cell to keep the volume of the receptor solution constant. The samples were filtered using a cellulose acetate membrane (0.45 μm of pore size, ADVANTEC TOYO KAISHA, LTD.). The concentrations of the WT1 peptide and R848 were measured by a HPLC system equipped with a UV detector (LC-2010C HT; SHIMAZU) to calculate the accumulative skin permeation amounts thereof. The HPLC condition was as follows: Column: YMC Pack Pro C18 RS 5 μm, 4.6×150 mm; YMC CO., LTD., Mobile Phase: acetonitrile/0.1% trifluoroacetic acid (23/77, v/v) at 35° C., Flow Rate: 1.0 mL/min. The wavelength of the UV detector was set to 215 nm. The retention times of the WT1 peptide and R848 were about 7.2 minutes and 4.6 minutes, respectively.

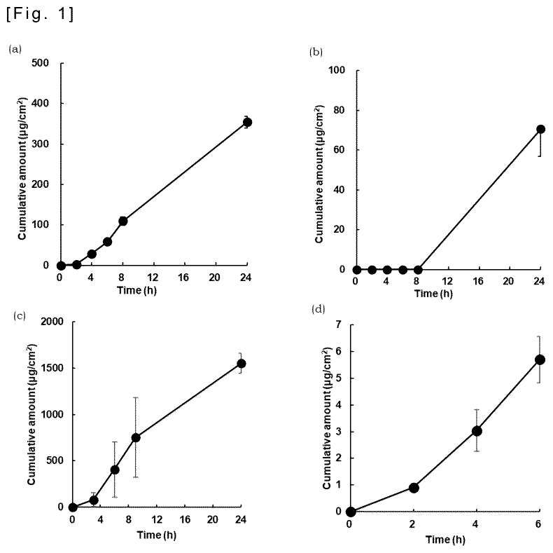

The accumulative skin permeation amounts of the WT1 peptide and R848 in Preparation Example 1 after 2, 4, 6, 8 and 24 hours (μg/cm²), the accumulative skin permeation amounts of the WT1 peptide in Preparation Example 2 after 2, 4 and 6 hours (μg/cm²) and the accumulative skin permeation amounts of R848 in Preparation Example 3 after 3, 6, 9 and 24 hours (μg/cm²) are shown in Table 2. In addition, the change in the accumulative skin permeation amount of R848 in Preparation Example 1 is shown in FIG. 1(a), the change in the accumulative skin permeation amount of the WT1 peptide in Preparation Example 1 is shown in FIG. 1(b), the change in the accumulative skin permeation amount of R848 in Preparation Example 3 is shown in FIG. 1(c), and the change in the accumulative skin permeation amount of the WT1 peptide in Preparation Example 2 is shown in FIG. 1(d).

TABLE 2

| | Preparation | Preparation | | |
|---|---|---|---|---|
| | Ex.1 | Ex. 1 | Preparation | Preparation |
| | (WT1 peptide) | (R848) | Ex.2 | Ex.3 |
| Accumulative | 0 | 3.05 | 0.91 | 81.6 (3h) |
| skin permeation | | | | |
| amount after 2 h | | | | |
| Accumulative | 0 | 29.7 | 3.04 | |
| skin permeation | | | | |
| amount after 4 h | | | | |
| Accumulative | 0 | 58.6 | 5.7 | 408.2 |
| skin permeation | | | | |
| amount after 6 h | | | | |
| Accumulative | 0 | 110 | | 751.5 (9h) |
| skin permeation | | | | |
| amount after 8 h | | | | |
| Accumulative | 70.6 | 354.3 | | 1549.2 |
| skin permeation | | | | |
| amount after 24 h | | | | |

It was shown that the transdermal absorption rate of the WT1 peptide when the WT1 peptide was individually administered with the ionic liquid in Preparation Example 2 and the transdermal absorption rate of R848 when the R848 was individually administered with the ionic liquid in Preparation Example 3 were faster (FIGS. 1(d) and (c)), as compared with each transdermal absorption rate of the WT1 peptide and R848 when the WT1 peptide and R848 were co-administered with the ionic liquid in Preparation Example 1 (FIGS. 1(a) and (b)).

The accumulative amount of the co-administered R848 for 24 hours was about 350 μg/cm², but the accumulative amount of the individually-administered R848 for 24 hours was about 1500 μg/cm². The permeability ratio of the WT1 peptide was lower and slower than that of R848. When the WT1 peptide and R848 were co-administered, no absorption of the WT1 peptide was almost observed even after 8 hours, whereas when the WT1 peptide and R848 were individually administered, the absorption of the WT1 peptide was observed after 2 hours. Hence, it was shown that when the WT1 peptide and R848 were individually administered, optimal ionic liquids could be used for the transdermal absorption of each of the WT1 peptide and R848 and the transdermal absorption rates thereof could be enhanced.

### Example 3: Study of Effect of Inducing CTL in WT1 Peptide/R848 Preparation

The abdomen or back of C57BL/6N mice was shaved or removed with a hair removal tape (n=3). An ionic liquid-containing preparation patch comprising the WT1 peptide (10 μg) and R848 (10 μg) (with the same composition as Preparation Example 1) was applied to the abdominal or back skin of each mouse once a week and the patch was removed from the skin after 24 hours. Such treatment was repeated three times. In the positive control, the WT1 peptide emulsified with complete Freund's adjuvant (CFA) was subcutaneously injected to each mouse once a week. Such subcutaneous injection was repeated three times. After 1 week of each final administration, the spleen was removed and collected from each mouse. The collected spleen was suspended by a cell strainer (100 μm, Becton Dickinson, NJ, USA), and then red blood cells were removed with 0.83% ammonium chloride to give a spleen suspension. As the control, a spleen cell suspension from untreated mouse was used.

The WT1-specific CTLs were stained with T-Select H-2db WT1 Tetramer-RMFPNAPYL-PE and anti-mouse CD8-FITC (MBL) and analyzed by a flow cytometer (Gallios, Beckman Coulter).

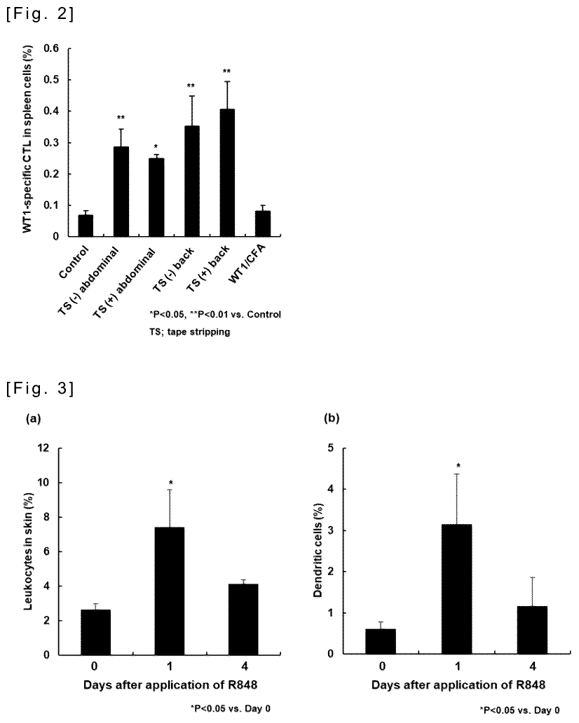

The ratios of cytotoxic T lymphocytes (CTLs) in spleen cells after the treatment with the control, the WT1 peptide/R848 preparations applied to abdomen (TS (−) and TS (+)) and back (TS (−) and TS (+)) and WT1/CFA preparation which is the positive control (%) are shown in FIG. 2. TS (−) shows that tape stripping treatment is not included, and TS (+) shows that tape stripping treatment is included.

FIG. 2 shows that the immunization with the WT1 peptide/R848 preparations significantly induced WT1-specific CTLs regardless of the presence of the stratum corneum. In addition, the applied sites of the preparations did not affect the induction of WT1-specific CTLs. The results showed that the use of an ionic liquid enhanced the permeability of the WT1 peptide and R848 into the stratum corneum and thereby enhanced the induction of WT1-specific CTLs.

### Example 4: Study of Effect of Inducing CTL in Combined Preparation of WT1 Peptide Preparation and R848 Preparation

An ionic liquid-containing preparation patch comprising R848 (10 μg) (with the same composition as Preparation Example 3) was applied to the shaved abdominal skin of C57BL/6N mice and the patch was removed from the skin after 1 day or 4 days of the application. The skin section to which the patch was applied was removed and collected, the collected skin section was cut into smaller sizes, and then the cut skin section was digested with collagenase 4 (Worthington Biochemical, NJ, USA) and DNase (Roche Diagnostic, Mannheim, Germany) at 37° C. for 120 minutes. After the digestion, cells were collected by filtration using a cell strainer (100 μm, Becton Dickinson, NJ, USA). The collected cells were stained with PE-labeled anti-mouse CD45 antibody (Miltenyi Biotec, Bergisch Gladbach, Germany) and APC-labeled anti-mouse CD11c antibody (BioLegend, CA, USA), and white blood cells and dendritic cells in the skin section were identified. The dead cells were stained with 7-AAD (Becton Dickinson) and removed by flow cytometry.

In addition, an ionic liquid-containing preparation patch comprising R848 (10 μg) (with the same composition as Preparation Example 3) was applied to the shaved abdominal skin of each C57BL/6N mouse and the patch was removed from the skin after 24 hours of the application, and then an ionic liquid-containing preparation patch comprising WT1 peptide (10 μg) (with the same composition as Preparation Example 2) was applied to the same site and the patch was removed from the skin after 24 hours of the application. After 1 week, the spleen was removed from each mouse. According to a similar procedure to Example 3, a spleen cell suspension was prepared and the cytotoxic T lymphocytes in spleen cells were measured to evaluate the effect of inducing the WT1-specific CTLs. As the control, a spleen cell suspension from untreated mouse was used.

The ratios of white blood cells and dendritic cells after 0 day, 1 day and 4 days of the application of R848 preparation (%) are shown in FIG. 3. In FIG. 3, (a) shows the ratio of white blood cells (%), and (b) shows the ratio of dendritic cells (%).

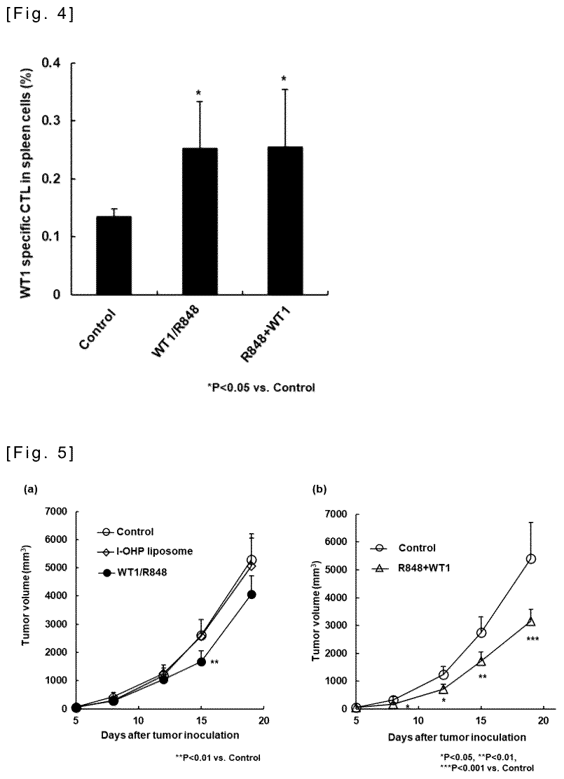

In addition, the ratios of cytotoxic T lymphocytes (CTLs) in spleen cells after the administration of the ionic liquid-containing preparation comprising WT1 peptide/R848 of Example 3 as well as the combined preparation of the ionic liquid-containing preparation comprising WT1 peptide and the ionic liquid-containing preparation comprising R848 of the present invention (%) are shown in FIG. 4.

FIG. 3 shows that white blood cells and dendritic cells in the skin were temporarily increased after 1 day of the application of the ionic liquid preparation comprising R848, but the temporary increase was not observed when applied for 4 days.

In addition, it was confirmed that the effect of inducing the CTLs was at the same level both when the WT1 peptide and R848 were co-administered and when R848 was administered and then the WT1 peptide was administered (FIG. 4).

### Example 5: Study of Anti-Tumor Effect of Combined Preparation of WT1 Peptide Preparation and R848 Preparation in C57BL/6N Mice Models Transplanted with LLC Cells

Firstly, C57BL/6N mice (20 mice) were randomly classified into the control group, the I-OHP-containing liposome treatment group, the WT1 peptide/R848 preparation treatment group and the combined preparation of WT1 peptide preparation and R848 preparation treatment group (n=5 per group).

Lewis lung carcinoma cell lines (LLC, 5×10⁵ cells) were subcutaneously inoculated to the back of each C57BL/6N mouse in each group (n=5). In the I-OHP-containing liposome treatment group, when the volume of tumor reaches 50 to 100 mm³ on the 6th day from the subcutaneous inoculation, I-OHP-containing liposome was intravenously injected to each mouse and then the similar administration was performed again on the 13th day. In the WT1 peptide/R848 preparation treatment group, the WT1 peptide/R848 preparation was applied to the abdominal skin of each mouse on the 7th day from the subcutaneous inoculation and the preparation was removed on the 8th day. Similar administration was performed again on the 14th day. In the combined preparation of WT1 peptide preparation and R848 preparation, R848 preparation was applied to the abdominal skin of each mouse on the 6th day from the subcutaneous inoculation and the preparation was removed on the 7th day, and then the WT1 peptide preparation was applied to the same site on the 7th day and the preparation was removed on the 8th day. Similar administration was performed again after 1 week. As the control, untreated mice were used.

The tumor volumes of mice in each group were measured twice a week using calipers. The tumor volume was calculated using the formula: Tumor Volume (mm³)=0.5×(Length)×(Width)².

The results of the measured tumor volume of mice in the control group, the I-OHP-containing liposome treatment group, the WT1 peptide/R848 preparation treatment group and the combined preparation of WT1 peptide preparation and R848 preparation treatment group (mm³) are shown in FIG. 5. In FIG. 5, (a) shows the results of the control group, the I-OHP-containing liposome treatment group and the WT1 peptide/R848 preparation treatment group, and (b) shows the results of the control group and the combined preparation of WT1 peptide preparation and R848 preparation treatment group.

The treatment with I-OHP-containing liposome had little effect on the growth of tumor. The treatment with WT1 peptide/R848 preparation significantly produced the effect of inhibiting the growth of tumor on 15th day. Also, there was a tendency to inhibit the growth of tumor on the 19th day, but the effect was not significant. The treatment with the combined preparation of WT1 peptide preparation and R848 preparation significantly produced the effect of inhibiting the growth of tumor on 8th day and remarkably produced the anti-tumor effect on the 19th day.

The results showed that the treatment with the combined preparation of WT1 peptide preparation and R848 preparation produced the better anti-tumor effect. Hence, the results suggest that the combined preparation of the present invention can be used as a preparation for transdermal administration in the treatment of cancer.

### Example 6: Study of Anti-Tumor Effect of OVA Peptide/R848 Preparation in C57BL/6N Mouse Models Transplanted with EG7-OVA Cells

Firstly, C57BL/6N mice (19 mice) were randomly classified into the control group (n=5), the I-OHP-containing liposome treatment group (n=4), the OVA peptide/R848 preparation treatment group (n=5) and the combination of I-OHP-containing liposome and OVA peptide/R848 preparation treatment group (n=5).

OVA-expressing T-lymphoma cell lines (EG7-OVA, 1×10⁶ cells) were subcutaneously inoculated to the back of each C57BL/6N mouse in each group. In the I-OHP-containing liposome treatment group, when the tumor volume reaches 50 to 100 mm³ on the 6th day from the subcutaneous inoculation, I-OHP-containing liposome was intravenously injected to each mouse. In the OVA peptide/R848 preparation treatment group, OVA peptide/R848 preparation was applied to the abdominal skin of each mouse on the 7th day from the subcutaneous inoculation and the preparation was removed on the 8th day. Similar administration was performed again on the 14th day. As the control, untreated mice were used.

The tumor volume of mice in each group was measured twice a week using calipers. The tumor volume was calculated using the formula: Tumor Volume (mm³)=0.5×(Length)×(Width)².

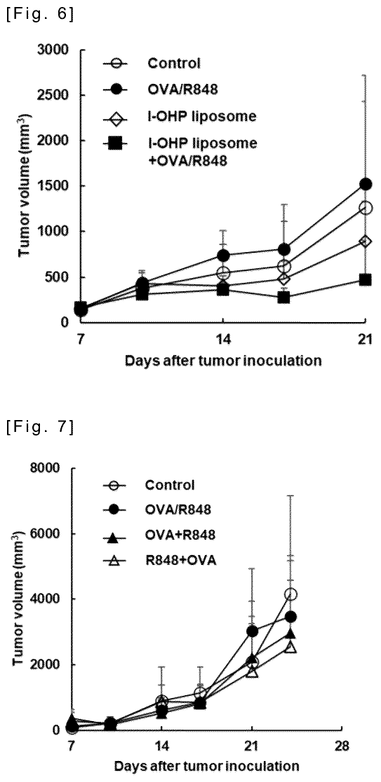

The results of the measured tumor volume of mice in the control group, the I-OHP-containing liposome treatment group, the OVA peptide/R848 preparation treatment group and the combination of I-OHP-containing liposome and OVA peptide/R848 preparation treatment group (mm³) are shown in FIG. 6.

The treatment with I-OHP-containing liposome alone and the treatment with OVA peptide/R848 preparation alone had little effect on the growth of tumor. In the combined treatment of I-OHP-containing liposome and OVA peptide/R848 preparation, there was a tendency to inhibit the growth of tumor.

### Example 7: Study of Anti-Tumor Effect of OVA Peptide Preparation and R848 Preparation in C57BL/6N Mouse Models Transplanted with EG7-OVA Cells

Firstly, C57BL/6N mice (18 mice) were randomly classified into the control group (n=5), the OVA peptide/R848 preparation treatment group (n=5), the combined preparation of OVA peptide preparation and R848 preparation treatment group (n=4) and the combined preparation of R848 preparation and OVA peptide preparation treatment group (n=4).

OVA-expressing T-lymphoma cell lines (EG7-OVA, 10⁶ cells) were subcutaneously inoculated to the back of each C57BL/6N mouse in each group. In the OVA peptide/R848 preparation treatment group, OVA peptide/R848 preparation was applied to the abdominal skin of each mouse on the 7th day from the subcutaneous inoculation and the preparation was removed on the 8th day. Similar administration was performed again on the 14th day. In the combined preparation of OVA peptide preparation and R848 preparation treatment group, the OVA peptide preparation was applied to the abdominal skin of each mouse on the 7th day from the subcutaneous inoculation and the preparation was removed on the 8th day, and then the R848 preparation was applied to the same site on the 8th day and the preparation was removed on the 9th day. Similar administration was performed again after 1 week. In the combined preparation of R848 preparation and OVA peptide preparation treatment group, the R848 preparation was applied to the abdominal skin of each mouse on the 7th day from the subcutaneous inoculation and the preparation was removed on the 8th day, and then the OVA peptide preparation was applied to the same site on the 8th day and the preparation was removed on the 9th day. Similar administration was performed again after 1 week. As a control, untreated mice were used.

The tumor volume of mice in each group was measured twice a week using calipers. The tumor volume was calculated using the formula: Tumor Volume (mm³)=0.5×(Length)×(Width)².

The results of the measured tumor volume of mice in the control group, the OVA peptide/R848 preparation treatment group, the combined preparation of OVA peptide preparation and R848 preparation treatment group and the combined preparation of R848 preparation and OVA peptide preparation treatment group (mm³) are shown in FIG. 7.

Among all of the groups, the tumor volume of mice in the combined preparation of R848 preparation and OVA peptide preparation treatment group was the smallest, and it was observed that the effect of inhibiting the growth of tumor in the group was more remarkable as compared to the control group.

The results showed that the combined preparation of R848 preparation and OVA peptide preparation produced a better anti-tumor effect as with the combined preparation of R848 preparation and WT1 peptide preparation.

### Example 8: Study of Skin Immune Activation with Combined Preparation of R848 Preparation and OVA Peptide Preparation

An ionic liquid-containing preparation patch comprising R848 (100 μg) (with the same composition as Preparation Example 3) was applied to the shaved abdominal skin of each C57BL/6N mouse and the patch was removed from the skin after 1 day. An ionic liquid-containing preparation patch comprising OVA peptide (100 μg) (with the same composition as Preparation Example 5) was then applied to the same site and the patch was removed from the skin after 0, 1, 3, 6 or 12 hours. The skin section to which the patch was applied was removed and collected, the collected skin section was cut into smaller sizes, and then the cut skin section was digested with collagenase 4 (Worthington Biochemical, NJ, USA) and DNase (Roche Diagnostic, Mannheim, Germany) at 37° C. for 120 minutes. After the digestion, cells were collected by filtration using cell strainer (100 μm, Becton Dickinson, NJ, USA). The collected cells were stained with FITC-labeled anti-mouse CD45 antibody (Invitrogen, CA, USA), APC-labeled anti-mouse CD11c antibody (BioLegend, CA, USA), and PE-labeled anti-mouse H-2Kb⋅SIINFEKL complex antibody (BioLegend, CA, USA), and the number of white blood cells, the number of dendritic cells and the amount of H-2Kb⋅SIINFEKL complex (antigen presentation level of OVA peptide) in the skin section were measured by a flow cytometer. The dead cells were stained with 7-AAD (Becton Dickinson) and removed by a flow cytometry.

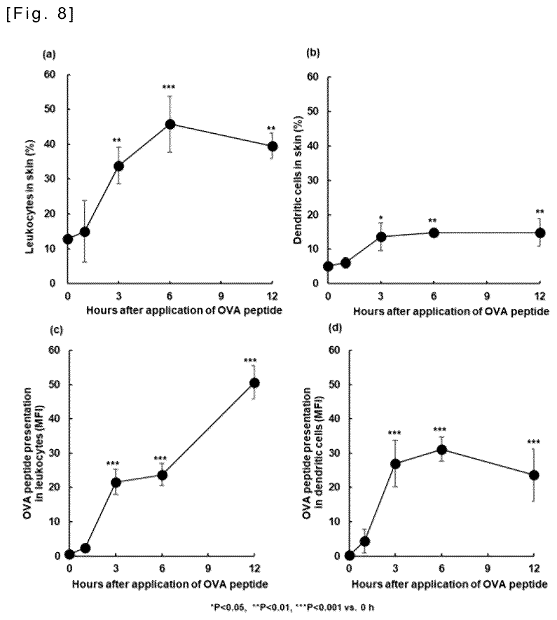

The results of the ratio of immune cells in the skin tissue (%) and the antigen presentation level of OVA peptide (Mean Fluorescence Intensity; MFI) in the treatment with the combined preparation of R848 preparation and OVA peptide preparation are shown in FIG. 8. In FIG. 8, (a) shows the ratio of white blood cells (%), (b) shows the ratio of dendritic cells (%), (c) shows the antigen presentation level of OVA peptide in white blood cells (MFI), and (d) shows the antigen presentation level of OVA peptide in dendritic cells (MFI).

FIG. 8 shows that the increases of white blood cells and dendritic cells in the skin were observed from 3 hours after the application of the OVA peptide preparation and the cell numbers reached equilibrium after 6 hours. In addition, it was confirmed that OVA peptide was presented as an antigen in white blood cells and dendritic cells after 3 hours of the application of OVA peptide preparation.

The results showed that the delivery of immune cells into the skin was enhanced and then OVA peptide was promptly presented as an antigen by the administration of the combined preparation of R848 preparation and OVA peptide preparation.

### Example 9: Study of Immune Cell Activation in Lymph Nodes with Combined Preparation of R848 Preparation and OVA Peptide Preparation

An ionic liquid-containing preparation patch comprising R848 (100 μg) (with the same composition as Preparation Example 3) was applied to the shaved abdominal skin of each C57BL/6N mouse and the patch was removed from the skin after 1 day. An ionic liquid-containing preparation patch comprising OVA peptide (100 μg) (with the same composition as Preparation Example 5) was then applied to the same site and the patch was removed from the skin after 0 or 6 hours. The inguinal lymph nodes and axillary lymph nodes, which are regional lymph nodes, were collected and the collected lymph nodes were suspended by a cell strainer (100 μm, Becton Dickinson, NJ, USA), and then red blood cells were removed with 0.83% ammonium chloride to give a lymph node suspension. As the control, a lymph node cell suspension from untreated mouse was used.

The collected cells were stained with FITC-labeled anti-mouse CD11b antibody (Invitrogen, CA, USA), APC-labeled anti-mouse CD11c antibody (BioLegend, CA, USA), PE-labeled anti-mouse CD86 antibody (Invitrogen, CA, USA), and PE-labeled anti-mouse H-2Kb SIINFEKL complex antibody (BioLegend, CA, USA), and the number of macrophage cells and the number of dendritic cells in the lymph nodes as well as the level of H-2Kb⋅SIINFEKL complex (antigen presentation level of OVA peptide) and the expression level of CD86 (Co-stimulatory molecule) in each cell were measured by a flow cytometry.

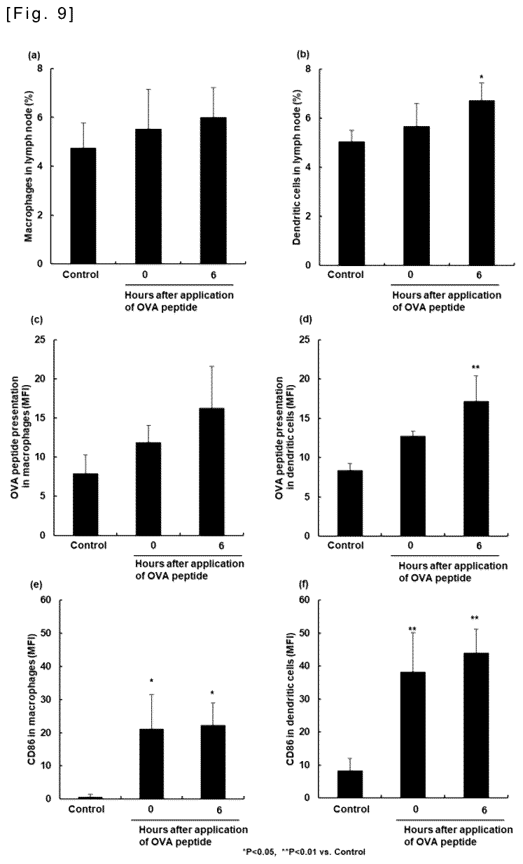

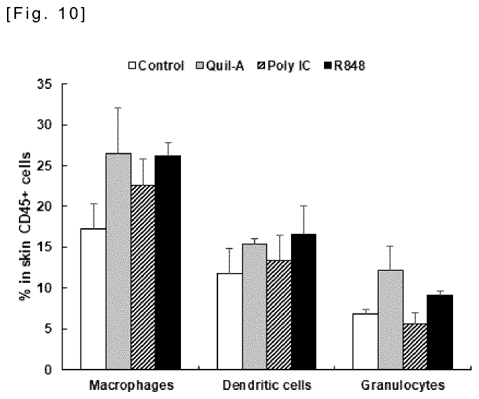

The results of the ratios of immune cells in lymph nodes (%), the antigen presentation levels of OVA peptide in immune cells (MFI) and the expression levels of CD86 in immune cells (MFI) in the control (untreatment) and the treatment with the combined preparation of R848 preparation and OVA peptide preparation are shown in FIG. 9. In FIG. 9, (a) shows the ratio of macrophage cells (%), (b) shows the ratio of dendritic cells (%), (c) shows the antigen presentation level of OVA peptide in macrophage cells (MFI), (d) shows the antigen presentation level of OVA peptide in dendritic cells (MFI), (e) shows the expression level of CD86 in macrophage cells (MFI), and (f) shows the expression level of CD86 in dendritic cells (MFI).

The dendritic cells in regional lymph nodes were slightly increased after 6 hours of the application of the OVA peptide preparation. Also, in the dendritic cells, the antigen presentation of OVA peptide was observed. In addition, in the macrophage cells and dendritic cells, the expression level of CD86, which is a co-stimulatory molecule, was increased.

The results showed that immune cells were activated in the lymph nodes and the antigen presentation was enhanced by the administration of the combined preparation of R848 preparation and OVA peptide preparation.

The above results suggest that the administration of the combined preparation of an antigenic peptide preparation and an adjuvant preparation migrated immune cells into the skin to incorporate antigenic peptides, and also delivered the immune cells to the lymph nodes and then produced the activation of the immune cells and the enhancement of the antigen presentation in the lymph nodes to ultimately induce cytotoxic T cells.

### Example 10: Study of Each Adjuvant with an Immune Cell Migration Effect to the Skin

In this study, each ionic liquid-containing preparation patch comprising R848, Poly-IC (R&D systems, MN, USA) or Quil-A (Invivogen, CA, USA) as an adjuvant was applied to the skin and an immune cell migration effect of the adjuvant to the skin was evaluated.

Specifically, an ionic liquid-containing preparation patch comprising R848 (100 μg) (with the same composition as Preparation Example 3) and the ionic liquid-containing preparation patches with same composition as Preparation Example 3 in which Poly-IC (100 μg) or Quil-A (100 μg) is used in place of R848 (100 μg) were applied to each shaved abdominal skin of each C57BL/6N mouse, and the patches were removed from the skin after 1 day of the application. The skin section to which the patch was applied was removed and collected, the collected skin section was cut into smaller sizes, and then the cut skin section was digested with collagenase 4 (Worthington Biochemical, NJ, USA) and DNase (Roche Diagnostic, Mannheim, Germany) at 37° C. for 120 minutes. After the digestion, cells were collected by filtration using cell strainer (100 μm, Becton Dickinson, NJ, USA). The collected cells were stained with FITC-labeled anti-mouse CD45 antibody (Invitrogen, CA, USA), PE-labeled anti-mouse CD11b antibody (BioLegend, CA, USA), APC-labeled anti-mouse CD11c antibody (BioLegend, CA, USA) or PE-labeled anti-mouse Gr-1 antibody (Invitrogen, CA, USA), and the numbers of macrophages, dendritic cells and granulocytes in the skin section were measured with a flow cytometer. The dead cells were stained with 7-AAD (Becton Dickinson) and removed by a flow cytometry.

The ratios of each type of immune cells (macrophage cells, dendritic cells and granulocytes) in CD45 positive cells in the skin tissue in the control (untreatment) and the treatment with each preparation comprising an adjuvant (R848, Poly-IC or Quil-A) (%) are shown in FIG. 10.

The preparation comprising R848, the preparation comprising Poly-IC and the preparation comprising Quil-A increased the macrophage cells and dendritic cells in the skin tissue after 1 day of the application of each preparation. The preparation comprising R848 and the preparation comprising Quil-A also increased the granulocytes.

The results shows that the invasion of various immune cells into the skin can be enhanced by preliminary applying R848, Poly-IC and Quil-A to the skin. Hence, it is suggested that Poly-IC and Quil-A can be used as an adjuvant for producing the similar effect to R848.

### INDUSTRIAL APPLICABILITY

According to the present invention, an antigenic peptide and an adjuvant effectively penetrate the skin and thus the antigenic peptide can be presented to antigen presenting cells (APCs) to significantly activate the immune response. For example, when cancer antigenic peptide is used as an antigenic peptide, the remarkable induction of cytotoxic T lymphocytes can be achieved systemically and thus the peptide can be used in the treatment of cancer such as skin cancer and lung cancer. In addition, the effect of enhancing the immune response with an adjuvant can be improved by administering a preparation for transdermal administration comprising an antigenic peptide and a preparation for transdermal administration comprising an adjuvant separately, and thus the effect of activating the immune response with the antigenic peptide can be further enhanced. The combined preparation of the present invention is expected as a preparation for transdermal administration for use in vaccines.

1. A combined preparation comprising a first preparation for transdermal administration comprising an antigenic peptide and an aliphatic carboxylic acid-based ionic liquid, and a second preparation for transdermal administration comprising an adjuvant and an aliphatic carboxylic acid-based ionic liquid.

2. The combined preparation according to claim 1, wherein the second preparation for transdermal administration is applied to the skin and then the first preparation for transdermal administration is applied to the same site.

3. The combined preparation according to claim 1, wherein the antigenic peptide is cancer antigenic peptide.

4. The combined preparation according to claim 3, wherein the cancer antigenic peptide is WT1 peptide.

5. The combined preparation according to claim 1, wherein the adjuvant is Resiquimod, Poly-IC or Quil-A.

6. The combined preparation according to claim 1, wherein the aliphatic carboxylic acid-based ionic liquid is an aliphatic carboxylic acid-based mixed ionic liquid.

7. The combined preparation according to claim 6, wherein the aliphatic carboxylic acid-based mixed ionic liquid is prepared from a) one or more lower aliphatic carboxylic acid-based ionic liquids selected from salts of ethanolamine, diethanolamine or triethanolamine with a short-chain fatty acid and b) an aliphatic carboxylic acid-based ionic liquid having 2 to 20 carbon atoms.

8. The combined preparation according to claim 1, wherein each of the first and second preparations for transdermal administration further comprises an excipient.

9. The combined preparation according to claim 1, which is a preparation for vaccine.

10. The combined preparation according to claim 3, which is an agent for inducing cytotoxic T lymphocytes.

11. Use of a first preparation for transdermal administration comprising an antigenic peptide and an aliphatic carboxylic acid-based ionic liquid as well as a second preparation for transdermal administration comprising an adjuvant and an aliphatic carboxylic acid-based ionic liquid in the manufacture of an agent for vaccine.

12. The use according to claim 11, wherein the agent for vaccine is an agent for cancer vaccine and the antigenic peptide is cancer antigen peptide.

13. A kit of preparation for transdermal administration comprising:
1) a first preparation for transdermal administration comprising an antigenic peptide and an aliphatic carboxylic acid-based ionic liquid;
2) a second preparation for transdermal administration comprising an adjuvant and an aliphatic carboxylic acid-based ionic liquid; and
3) instructions for use for administering the above 1) and 2) in combination.

14. The kit according to claim 13 for use as cancer vaccine, wherein the antigenic peptide is cancer antigen peptide.
